(19) United States
(12) Patent Application Publication
(10) Pub. No.: US 2005/0163825 A1
(43) Pub. Date: Jul. 28, 2005

(54) Lactoferrin-treated filament materials

(76) Inventor: A. Satyanarayan Naidu, Diamond Bar, CA (US)

Correspondence Address: KENNETH J. HOVET;NORDMAN, CORMANY, HAIR & COMPTON, P.O. BOX 9100, 1000 TOWN CENTER DRIVE, OXNARD, CA, 93031-9100, US

(21) Appl. No.: 10/766,783

(22) Filed: Jan. 27, 2004

(51) Int. Cl. A61K 38/40; A61K 48/00

(52) U.S. Cl. 424/443; 514/6; 514/2; 514/44; 514/54; 514/56

(57) ABSTRACT

Disclosed is a filament composition having a surface for reducing microbial contamination having a surface for reducing microbial contamination comprising a filament material, such as a dental floss or a suture material, and lactoferrin.

Throughout this application various publications are referenced. The disclosures of these publications in their entireties are hereby incorporated by reference in this application in order to more fully describe the state of the art to which this invention pertains.

### BACKGROUND OF THE INVENTION

1. Field of the Invention

This invention relates to the chemical arts. In particular, it relates to treated filament materials having antimicrobial properties.

2. Discussion of the Related Art

Once pathogenic microbes invade the natural barriers of the body they present the risk of infection to the invaded host. Two common means of access by bacteria are through the oral cavity, potentially causing a variety of periodontal disease and at the site of sutured tissue, as in the case of wounds and/or surgical intervention sites, where the body's normal immune defenses are breached.

The teeth and the mouth are vulnerable to many diseases, infections, and disorders. It is well known that a good regime of oral hygiene is the best preventative measure against cavities or dental caries, gum or periodontal disease, viral and fungal infections of the oral cavity, and other dental disorders.

Gum disease is an infection of the tissues surrounding and supporting the teeth. It is a major cause of tooth loss. In the early stages of gum disease, called gingivitis, the gums can become red, swollen and bleed easily. At this stage the disease is still reversible and can usually be eliminated by brushing and flossing.

Periodontitis is the more advanced stage of gum disease in which the gums and bones that support the teeth become severely damaged. Periodontitis can be caused by unremoved plaque. Plaque is a film of bacteria and mucous that grows on the tooth surface. Some of the bacteria in the plaque make acids which cause tooth decay. Other kinds of bacteria in the plaque make toxins which cause gum disease. The plaque causes the gums to become irritated and inflamed. The irritated gum tissue can separate from the teeth and form spaces called pockets. Bacteria move into the pockets and continue to cause irritation. Left untreated the process can continue until the bone and other tooth-supporting tissues are destroyed. Various agents are currently used to control plaque formation and other microbial infections in the mouth, but, unfortunately, they suffer from a variety of drawbacks.

Flossing is an extremely important component of proper dental hygiene. Dental flosses have long been used effectively to clean the spaces between the teeth and under the gum margin. One example of a dental floss is disclosed in U.S. Pat. No. 3,800,812. The art of present commercial dental flosses is well exemplified by U.S. Pat. No. 4,414,990, U.S. Pat. No. 4,033,365 and U.S. Pat. No. 3,943,949 which disclose the use of various non-polytetrafluoroethylene filaments as a floss; U.S. Pat. No. 5,033,488 which discloses a floss with a single strand of expanded polytetrafluoroethylene that has been coated with a microcrystalline wax; and U.S. Pat. No. 6,270,890 which discloses a dental floss of both polytetrafluoroethylene filaments and non-polytetrafluoroethylene filaments.

To increase the effectiveness of the floss, some flosses have included certain medicinal ingredients, such as fluoride compounds, to protect the tooth enamel from acid attack. For example, U.S. Pat. No. 3,830,246, U.S. Pat. No. 3,897,795, U.S. Pat. No. 4,215,478 and U.S. Pat. No. 3,771,536 disclose dental flosses which include a fluoride compound to aid in the delivery of the fluoride to the tooth surface between adjacent teeth. Bactericides have also been used in connection with dental floss to inhibit periodontal disease. For example, U.S. Pat. No. 6,159,447 and U.S. Pat. No. 6,482,396 disclose compositions for treating bacterial colonization and diseases in an oral cavity. The medicinal components have typically been applied as a coating to the dental floss.

It is also known to have dental flosses that include other kinds of ingredients. For example, U.S. Pat. No. 4,033,365 discloses a floss designed to retain flavorants over a long period of time through the use of non-wax polymeric coatings containing spray-dried flavor particles. U.S. Pat. No. 3,943,949 discloses a dental floss-like material in the form of a bundle of natural or synthetic fibers, such as nylon. The floss is coated with various waxes, including microcrystalline wax, to reduce the friction of the floss against the tooth surface. The wax coating is disclosed as containing a spray-dried flavorant to be dispersed during use.

Postoperative surgical site infections occur in approximately 2.5% of all patients who undergo surgical procedures. It is quite common that some type of suture is utilized. While the body's immune response normally is successful in preventing microbial infection at the wound site, in the presence of foreign matter, such as the suture, the probability of infection increases significantly.

The primary mode of infection associated with a suture is attachment of microorganisms, e.g., bacteria, to the suture, followed by their growth and proliferation on the suture. Subsequent release and migration of the microbial contaminant from the microbe's original attachment and growth to tissue immediate to and surrounding the suture results in an infection associated with the suture. Once the microbes attach and establish themselves on the suture, it is practically impossible to treat the infection without actually removing and replacing the suture or other wound closure material or device.

While antimicrobial substances bacteriocins, i.e., substances that in and of themselves are toxic to microorganisms capable of causing infection at surgical sites, may be added to suture, they typically have limitations. Many of the antimicrobial substances are toxic to the patient, while others cause allergic reactions. In addition, certain microorganisms are resistant to such antimicrobial substances due to the development of defense mechanisms that actually destroy the antimicrobial molecule.

Various products for use externally or internally with humans or animals can serve to introduce bacterial, viral, fungal or other undesirable infections. Such products include suture, medical devices, surgical gloves and implements, catheters, implants and other medical implements. To prevent such contamination, such devices can be treated with an antimicrobial agent. Known methods of preparing infection-resistant medical devices have been proposed in U.S. Pat. Nos. 3,566,874; 3,674,901; 3,695,921; 3,705,938; 3,987,797; 4,024,871; 4,318,947; 4,381,380; 4,539,234; 4,612,337; 3,699,956; 4,054,139; 4,592,920; 4,603,152; 4,667,143 and 5,019,096. U.S. Pat. No. 5,607,681 discloses anti-microbial compositions that can be impregnated on sutures and dental floss.

Antimicrobial agents with selective toxicity for a specific spectrum or range of pathogenic microorganisms are well known in the art. One class of antimicrobial agents is the antibiotics, which are compounds, synthesized and excreted by various microorganisms, that are selectively toxic to other microorganisms, specifically bacteria. In addition, some antibiotics can be artificially modified to produce antimicrobial agents that are more effective and/or more able to overcome antibiotic resistance.

PCT/US00/14818 describes an antimicrobial assay using buffered solutions containing both free lactoferrin and lactoferrin immobilized on a variety of substrates to block attachment (microbial blocking activity) of various oral pathogens to subepithelial matrix proteins, oromucoid cell line, hydroxyapatite (HA) and denatured bases acrylic resin (DBAR) surfaces. Lactoferrin is an antimicrobial iron-binding glycoprotein present in milk and various mammalian secretions (including saliva, tears, mucus, and seminal fluids). Crystallographic studies of LF indicate a bilobate structure (N-terminus and C-terminus lobes) with one iron-binding site in each lobe. LF has ability to reversibly bind two Fe³⁺ ions per lobe in coordination with two CO₃²⁻ ions. LF can release the bound iron in a fully reversible manner, either on exposure to lowered pH (below 4.0) or on receptor binding. This high affinity for iron is linked to many of its biological functions, including antimicrobial effects. Various laboratory studies have reported that the structural integrity of LF is critical for its antimicrobial effects against bacteria, fungi, protozoa, and viruses.

However, the activity of LF, like the activity of most proteins, is highly dependent on the three-dimensional or tertiary structure of the protein. If the protein does not have the proper conformation its activity is diminished or lost. LF's instability limits it usefulness. Consequently, before LF can be used for commercial application, it would be expected to become denatured or inactivated, and lose its antimicrobial properties.

### SUMMARY OF THE INVENTION

Now, in accordance with the invention there has been found a filament composition having a surface for reducing microbial contamination comprising a filament material, such as a dental floss or a suture material, and lactoferrin. In preferred embodiments, at least some of the lactoferrin is immobilized on a biologically active substrate via the N-terminus region of the lactoferrin. Preferably, the ratio of immobilized LF to free LF is from about 1:1 to about 1:500, more preferably from about 1:4 to about 1:100, and most preferably about 1:20. The concentration of the LF on the surface of the filament composition for reducing microbial contamination is typically from about 0.0001 to about 10 mg/square inch and preferably from about 0.01 to about 1 mg/sq. inch.

Representative filament materials include monofilament materials, multifilament materials and tapes. Representative biologically active substrates include proteins, polysaccharides, nucleic acids, nucleotides or lipids. Examples of preferred biologically active substrates include collagen, gelatin, fibronectin, casein, mucin, heparan-sulfate, carrageenan, pectin, deoxyribonucleic acid, adenosine triphosphate or triglycerides.

In some embodiments, the surface of the filament composition has a coating containing the lactoferrin. In other embodiments, the filament material is covalently bonded to the lactoferrin.

In some embodiments, the filament material is a dental floss material having a pH sensitive wax or polymeric coating. In other embodiments, the filament material is a dental floss material having a layer of a hydrophilic polymer treated with the lactoferrin and a permeation enhancer.

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

### DETAILED DESCRIPTION OF THE PREFERRED EMBODIMENTS

In accordance with the present invention there has been discovered filament materials that have been treated with LF. Representative filament materials include dental floss materials and suture materials. In preferred embodiments, at least some of the LF is immobilized on a substrate, via attachment of its amino-terminus (N-terminus), leaving its carbon-terminus (C-terminus) free.

Any suitable dental floss or suture material can be used in accordance with the invention. Useful dental flosses come in different forms and include monofilaments, multi-filaments, as well as tapes. As used in the present invention the term filament material includes such monofilaments, multi-filaments, and tape materials and the terms “dental floss” and “dental floss material” are meant to include all of the filamentous forms of floss, the tape forms of dental floss and equivalents of the same.

As a tape, the dental floss typically has a denier of about 1200 to 3000 or more. As multi-filaments, the individual filaments typically have a denier of about 100 to 800. The advantage of a multi-filament over a tape or a monofilament is that, in use, the filaments of a multi-filament floss splay and assist in the removal of food particles, debris and plaque from between the teeth and under the gum line. This enhanced cleaning comes from the splayed filaments each rubbing the surface of a tooth. The use of a plurality of filaments appears to exhibit an increased removal of certain particles and plaque.

In preferred embodiments, the dental floss material, as it is formed, will undergo a twisting to form the filaments into a more cohesive form. There can be from about one to five twists per inch of filament.

In some embodiments, the filament material has a coating containing the LF. Suitable coating formulation(s) can advantageously contain, in addition to LF, an appropriate carrier (s). For example, the skilled practitioner can employ as a carrier a non-toxic polymeric resin, additionally containing an effective amount of LF, which resin can be used to coat the surface of the treated filament material, hardening in place upon it.

In some embodiments, the suture material is coated with a coating, preferably a pH sensitive coating, which will release the LF under conditions of use. Representative coatings include wax and polymeric coatings, such as polyethylene coatings, or acrylic polymer coatings, including Eudragit-L or Eudragit-S coatings, or cellulose coatings, including ethyl cellulose coatings. Ethyl cellulose, for example, is amphoteric and dissolves to release the LF at either acidic or basic pH. Acrylic polymer coatings have various pH sensitivities. For example, Eudragit-S dissolves at above pH 7.0 to release the LF. Alternatively, the filament material can be sprayed, brushed or soaked with a wax coating material containing the LF.

Alternatively, the dental floss material can comprise an inner layer containing the LF, a permeation enhancer, such as a bile salt or fusidate, and a hydrophilic polymer, such as hydroxypropyl cellulose, hydroxypropyl methylcellulose, hydroxyethylcellulose, dextran, pectin, polyvinyl pyrrolidone, starch, gelatin, or any of a number of other polymers known to be useful for this purpose. This inner layer can have one surface adapted to contact and adhere to the moist mucosal tissue of the oral cavity and may have an opposing surface adhering to an overlying non-adhesive inert layer. Optionally, such dental floss material may be produced in a manner such that the inner layer also contains additional binding agents, flavoring agents or fillers. Some useful systems employ a non-ionic detergent along with a permeation enhancer. These examples are merely illustrative of available delivery technology that may be used with the dental floss material of the present invention and are not intended to represent of be limitations of the present invention.

The LF dental floss material of the present invention is optionally packaged on a spool, reel or disposed in a handle or other applicator. It may then be dispensed in any of the conventional manners.

Useful suture materials may be of any appropriate natural or synthetic composition. These materials may be used as single filament strands, i.e., monofilament sutures, or as multifilament strands in a braided, twisted or other multifilament construction. Examples of suitable materials include nylon, polypropylene, steel, polyvinyl fluoride, linen, cotton, silk and polyesters. Natural materials such as silk, cotton, linen, and the like, of course do not lend themselves to the fabrication of monofilament sutures and are accordingly generally used in one of the multifilament constructions.

Conventionally, sutures have been manufactured of non-degradable materials like nylon and polypropylene or degradable or absorbable materials like polyglycolic acid and copolymers of glycolic acid and lactic acid. Biodegradable or absorbable sutures are designed to decompose in the tissue environment of the patient, desirably after a time when wound recovery has occurred to an extent that their strength is no longer necessary. This allows patients to not have to return to a physician to have their suture removed. Absorbable sutures are manufactured from natural or synthetic materials. Some of the earliest absorbable sutures were made of natural material, such as collagenous material taken from sheep intestines. Such sutures are still in use today and are commonly referred to as “catgut” or simply “gut” sutures or ligatures. Gut sutures may be prepared in the form of threads or strands.

Absorbable sutures made from synthetic material are typically extruded in continuous lengths can be used in monofilament form. Common synthetic monofilament sutures include polyethylene terephthalate, polypropylene, polyethylene, polyglycolides, polylactides and nylon. Different circumstances for application of sutures demand different properties. Important properties that have to be adjusted, depending on the type of application of a suture, are knot strength, slip, tensile strength, pliability and the like. Such monofilament sutures are often preferred for many applications due to their inherent smoothness and noncapillarity to body fluids.

Absorbable sutures typically can be made for short term or long term use. The classification short term generally refers to sutures which retain at least about 20 percent of their original strength for three weeks, with the suture being essentially absorbed in the body within about 60 to 90 days. Absorbable multifilament sutures such as DEXON sutures (made from glycolide homopolymer and commercially available from Davis & Geck, Danbury, Conn.), VICRYL sutures (made from a copolymer of glycolide and lactide and commercially available from Ethicon, Inc., Sommerville, N.J.), and POLYSORB sutures (also made from a copolymer of glycolide and lactide and commercially available from United States Surgical Corporation, Norwalk, Conn.) are known in the industry as short term absorbable sutures. Long term absorbable sutures are generally classified retaining at least about 20 percent of their original strength for six or more weeks, with the suture being essentially absorbed in the body within about 180 days. For example, PDS II and MONOCRYL (commercially available from Ethicon, Inc., Sommerville, N.J.), MAXON suture (commercially available from Davis & Geck, Danbury, Conn.) and BIOSYN (commercially available from United States Surgical Corporation) are synthetic monofilament that reportedly generally fit a long-term absorption profile.

The terms “LF”, “LF protein”, and “LF peptide” are used interchangeably herein. The LF useful in accordance with the materials and methods of the present invention include or contain glycosylated or unglycosylated LF peptides. A full length LF peptide sequence has about 600 to about 800 contiguous amino acids. For example, native human LF is about 703 amino acids long; native bovine LF is about 651 amino acids long. Other useful mammalian LF sequences are of various but similar lengths. Useful LF peptides include full length native LF peptides and also include LF peptides lacking one to about eleven contiguous amino acids from the extreme end of the N-terminus region or the extreme end of the C-terminus region of a native LF peptide amino acid sequence. Also useful are LF peptides having sequences variant in one or more amino acid residues compared to a native LF sequence, but that remain at least partially functional. The term “functional”, when used herein as a modifier of LF protein (s) or peptide (s), generally refers to a polypeptide that retains the antimicrobial activity attributed to native LF amino acid sequences. In the context of immobilized LF, the term “functional”, when used herein as a modifier of LF protein (s) or peptide (s), generally refers to a polypeptide that exhibits both the ability to bind at its N-terminus to a substrate, i.e., become immobilized, and also retains the antimicrobial activity attributed to native LF amino acid sequences. Thus, the term LF encompasses functional LF having a variant amino acid sequence.

The LF can be, but is not necessarily, of homologous origin with respect to the vertebrate subject to which it is used or administered, in accordance with the present methods. Thus, for example, in accordance with the inventive method, LF of human origin functions to reduce or inhibit microbial contamination or growth in or on human and/or non-human vertebrates on sutured tissue surfaces, tooth and/or gum or other periodontal surfaces. Similarly, bovine LF can be used to treat either bovine or non-bovine subjects. However, for use on human or non-human vertebrates, in vivo, homologous LF is preferred to avoid adverse immunoreactions.

The LF peptide can be isolated from mammalian sources (humans, cows, sows, mares, transgenic animals, and the like), biological secretions, such as colostrum, transitional milk, matured milk, milk in later lactation, and the like, or processed products thereof such as skim milk and whey. Also useful for the isolation of LF is well-known recombinant DNA technology, whereby cloned LF-encoding genes are expressed in procaryotic and/or eucaryotic cells. The LF peptide is isolated by any conventional method, such as by chromatography, ion-exchanger, molecular-sieve or affinity column. Suitable LF also is commercially available from DMV International Nutritionals, the Netherlands; Morinaga Milk Company, Japan; BioPole, Belgium; and Glanbia, USA.

The LF can be immobilized on any suitable biologically active substrate, i.e., any substrate that can bind the N-terminus region of the LF without adversely affecting the LFs antimicrobial activity. In preferred embodiments, LF is immobilized on a naturally occurring substrate. Suitable substrates include proteins, polysaccharides, nucleic acids, nucleotides, and lipids. Preferred substrates include collagen, gelatin, fibronectin, casein, mucin, heparan-sulfate, carrageenan, pectin, deoxyribonucleic acid, adenosine triphosphate or a triglyceride.

Another preferred substrate is a galactose-rich polysaccharide (GRP). Galactose-rich polysaccharides are known in the art as water-soluble extracts of agar that contain a majority of galactose residues and/or galactose derivatives, which can be substituted or non-substituted. (Gerlach, D. et al., Identification of a novel lectin in Streptococcus pyogenes and its possible role in bacterial adherence to pharyngeal cells, Current Microbiology 28: 331-38 [1994]). Suitable galactose-rich polysaccharides include galactose derivatives comprising galactose, anhydrogalactose, 2-Ome-galactose, and 4-Ome-galactose, among others. Galactose-rich polysaccharides can also contain a minority of other sugar and non-sugar components, including residues of nitrogen-containing non-sugar compounds and/or sulfated residues. The galactose-rich polysaccharides can be purchased or extracted from commercial agars by known methods. (E.g., Gerlach, D. et al. [1994]; Naidu, A. S., Agar, Chapter 16, In: Natural Food Antimicrobial Systems, A. S. Naidu (ed.), CRC Press, Inc., pp. 417-27 (2000). Other suitable biologically active substrates include proteins, such as collagen, denatured collagen (gelatin), fibronectin, and casein; polysaccharides, such as mucin, heparan-sulfates, carrageenan, and cellulose; nucleic acids and their nucleotides, such as deoxyribonucleic acid and adenosine triphosphate; and lipids such as triglycerides.

The LF is immobilized on the substrate using any suitable technique. For example, LF can be immobilized simply by mixing isolated LF with the biologically active substrate in a suitable medium, such as deionized water. The immobilization process is dependent on the quality of the substrate as well as the quality of the LF. For example, in most of the commercially available lactoferrins, there is variation in the level of impurities (range: 4-20%), degree of non-specific cidal activity (range: 20-40%), and extent of protein denaturation (range: 10-25%). Consequently, the amount of substrate and the amount of LF to be used in the immobilization reaction will depend, inter alia, on the choice of starting materials. The immobilization technique and the amounts of substrate and LF are readily determined by a skilled artisan without undue experimentation.

In some embodiments, the immobilized LF (Im-LF) is combined with free LF. Mixtures of Im-LF and free LF are formed by adding excess LF to the substrate. Preferably, the ratio of the LF to free LF is from about 1:1 to about 1:500, more preferably from about 1:4 to about 1:100, and most preferably about 1:20.

The LF can be incorporated into the filament material by any suitable means, such as by including in a coating or a hydrophilic polymer layer, or by otherwise treating the filament material. In some embodiments, a covalent linkage between the filament material and the LF is produced. For example, some treatments result in conjugation of the LF to the polymeric material through, for example, diazo bonds or amide bonds. The filament material is treated at any time prior to the use. For example, the LF, preferably Im-LF, can be combined with the filament material during the manufacture of the suture or floss on the filament or filaments, prior to the twisting, braiding or other manufacturing treatment of the filaments.

LF may also be applied to the floss or suture material after its typical manufacture. An LF-containing coating formulation can be applied by any suitable method. Representative methods include spraying, brushing or soaking the filament material with a dispersion of the LF, including dispersions incorporated into a microsphere or particle (coated or not).

Dispersions useful in accordance with the invention can be prepared in various ways. A first way is by forming a solution containing the LF, most preferably an aqueous solution containing Im-LF, along with an emulsifier. Representative emulsifiers include mono-, di-triglyceride compounds, glycerol, phosphatidyl ethanolamine, phosphatidyl choline, or lecithin. One embodiment includes a mixture of mono- and diglyceride compounds. Suitable, commercially available mixtures (containing 35-45% monoglycerides) include GRUENAU Mono & Diglycerides, and C. G. 340-E (Bavaria Corporation, Altamonte Springs, Fla.). In embodiments in which LF is immobilized on a triglyceride or other lipid substrate, the Im-LF can be held in solution, if the solution has the properties of bile (i.e., is a solution of mixed micelles with bile salt added), or the solution contains a detergent or a solvent (e.g. the solution contains Tween).

A second way of preparing dispersions useful in accordance with the invention is by forming an emulsion containing the LF, i.e., by forming a two-phase system in which a first liquid, containing LF, is dispersed in the form of small globules throughout a second liquid that is immiscible with the first liquid. (Swinyard and Lowenthal, “Pharmaceutical Necessities” Remington's Pharmaceutical Sciences, 17^{th} ed., A R Gennaro (Ed), Philadelphia College of Pharmacy and Science, 1985, p. 1296). Aqueous emulsions containing a second, hydrophobic liquid phase are preferred. The concentration of LF in emulsions is typically from about 10.0 to about 0.001 wt. %, preferably from about 5.0 to about 0.05 wt. %, and more preferably from about 2.0 to about 0.2 wt. %.

A third way of preparing dispersions useful in accordance with the invention is by forming a suspension of a solid phase containing the LF, either dispersed within a liquid phase, such as a colloid suspension of LF, or dispersed among other solids (e.g., microcrystalline suspension), the composition thus having the form of a powder or a granular solid. The concentration of LF in such dispersions is typically from about 10.0 to about 0.001 wt. %, preferably from about 5.0 to about 0.05 wt. %, and more preferably from about 2.0 to about 0.2 wt. %.

In those embodiments where the dispersion is applied as a liquid spray, the dispersion can also contain from about 10.0 to about 0.001 wt. %, preferably from about 5.0 to about 0.05 wt. %, and more preferably from about 2.0 to about 0.2 wt. % of a film-forming agent. Suitable film-forming agents include carrageenan, gelatin or collagen (Type-I and Type-II).

Alternatively, the LF can be applied to the filament material, especially a polymeric dental floss material, by enmeshing, implanting, or impregnating the LF within the polymeric material, by means known to the artisan skilled in the art. In still other embodiments, the LF may be applied to the floss or suture material as they are dispensed for use. One skilled in the art will recognize that there are additional methods for treating the suture or dental floss material with the LF.

The surface of the inventive filament compositions contains an amount of LF effective to reduce microbial contamination. Preferably, the concentration of the LF on the surface for reducing microbial contamination is from about 0.0001 to about 10 mg/square inch., more preferably from about 0.01 to about 1 mg/sq. inch. This is sufficient concentration to inhibit the growth and/or adhesion of microbes on the surface contacted with the treated filament material.

The LF composition used to treat the filament material can include conventional additives. As is appropriate for the filament's use as suture or dental floss, representative additives may include one or more of the following, medicament(s), including additional antimicrobial agent(s), flavorant(s), nutrient(s), solvent(s), vehicle(s), adjuvant(s), excipient(s), binder(s), thickener(s), suspending agent(s), or filler substance(s). Useful additives include, but are not limited to, solid, semisolid or liquid glucose, lactose, sucrose, or polymeric substances like starch or dextran.

The additives can be applied to the filaments by any suitable method. Representative methods include applying the additive to the filament as a liquid and then drying the additive onto the filaments. Alternately, the additives can be applied to the filaments as a solid with the aid of a binder. Suitable binders include polyvinyl alcohol, and in particular, polyvinyl alcohol in combination with polyethylene glycol.

Useful additional antimicrobial agents include acid antimicrobials, such as lactic acid, acetic acid, citric acid, sorbic acids; ionic antimicrobials, such as polyphosphate, nitrites; sulfur compounds; chlorocides; ozone; or a natural, synthetic, or an semi-synthetic antibiotic agent, such as neomycin, metronidazole, teicoplanin, vancomycin, ciprofloxacin, doxycycline, tetracycline, augmentin, erythromycin, chloramphenicol, cephalexin (e.g., Keflex), penicillin, ampicillin, kanamycin, rifamycin, rifaximin, rifampin, clindamycin, trimethoprim, a 4-amino salicylate compound, a 5-aminosalicylate compound, a sulfonamide compound, a betalactam compound, an aminoglycoside compound, a macrolide compound, or a quinolone compound.

In dental floss compositions, a preferred form of flavorant is a spray dried flavorant. The flavorant can be essentially any flavor but is preferably a peppermint and/or spearmint. This can be applied to the filaments using a non-wax polymeric binder as is described in U.S. Pat. No. 4,033,365. If the dental floss composition is wax coated, the spray dried flavorant can be applied to the still molten wax.

The inventive compositions are useful against a wide variety of bacteria, such as, but not limited to pathogenic and non-pathogenic strains of:
(A) Gram-negative facultative anaerobes of the enteric group, for example, Escherichia coli; Helicobacter pylori; Salmonella spp., including Salmonella typhimurium, Salmonella typhi, Salmonella enteritidis, Salmonella abony, Salmonella dublin, Salmonella hartford, Salmonella kentucky, Salmonella panama, Salmonella pullorum, Salmonella rostock, Salmonella thompson, Salmonella virschow; Enterobacter spp., such as Enterobacter aerogenes; Klebsiella pneumonie; Shigella spp., such as Shigella dysenteriae or Shigella flexneri; Vibrio spp., including Vibrio cholerae; Yersinia enterocolitica and Yersinia pestis.
(B) Gram-negative aerobic motile rods, such as Bordetella pertussis; Campylobacter jejuni; and Pseudomonas spp., such as Pseudomonas aeruginosa;
(C) Gram-negative aerobic non-motile rods, such as Brucella spp.; Legionella pneumophila; and Francisella tularensis;
(D) Gram-positive bacteria, including coccoid forms such as Staphylococcus spp., such as Staphylococcus aureus, Staphylococcus epidermidis; Streptococcus spp., such as Streptococcus pyogenes, Streptococcus pneumoniae, Streptococcus mutans, Streptococccus sanguis; Pediococcus acne; and bacillary forms such as Bacillus spp., including Bacillus anthracis, Bacillus cereus, Bacillus pumilus, Bacillus subtilis; Clostridium spp., including Clostridium difficile, Clostridium tetani, Clostridium botulinum, Clostridium perfringens; and Listeria monocytogenes;
(E) Periodontal pathogens, such as Actinobacillus actinomycetemcomitans, Porphyromonas gingivalis; Prevotella spp., such as Prevotella intermedia.

Further, the inventive compositions are useful against fungal pathogens including dermatophytes, such as Epidermophyton spp.; Microsporium spp.; and Trichophyton spp.; systemic mycopathogens, such as Blastomyces spp.; Coccidiodes spp.; Cryptococcus neoformans; Histoplasma spp.; and yeasts, such as Candida albicans. Still further, the inventive compositions are useful against protozoan parasites, such as Entamoeba histolytica; Naegleria flowleri; Giardia lamblia; Leishmania spp.; Trichomonas vaginalis; Trypanosoma spp.; Plasmodium spp.; and Taxoplasma spp.

And still further, the inventive compositions and methods are also useful against viral pathogens, including herpes viruses, such as HHV-6 and HHV-8, Cytomegalovirus (CMV); Epstein-Barr virus (EBV); Herpes Simplex viruses (HSV); Varicella viruses; Picorna viruses such as Coxsackie viruses; Hepatitis A virus; Rhinoviruses; Retroviruses, such as the Rotaviruses, Influenza, and Parainfluenza viruses.

The inventive compositions are especially useful in treating or preventing infections, including clostridial infections, at any oral or periodontal site or tissue of a vertebrate. Such clostridial infections include, but are not limited to, gangrene or tetanus, caused, respectively, by Clostridium perfringens and Clostridium tetani, when these species grow in wounds and damaged tissues with low oxygen tension.

Moreover, the inventive compositions can act synergistically to potentiate some antibiotic agents, including beta-lactams, chloramphenicol, aminoglycosides, clindamycin, vancomycin, sulfonamides, trimethoprim, rifampin, tetracyclines, metronidazole, quinolones, erythromycin, and other macrolides.

The present invention includes a method of preventing or inhibiting the growth and/or adhesion of a microbe in or on a vertebrate subject, including a human subject. The human subject can be an infant, child, or adult. The method is also useful for veterinary purposes. The present method is useful for treating any non-human vertebrate including, but not limited to a wild, exotic, domestic, or farm animal. For example, the method is useful for treating an appropriate species of reptile, amphibian, avian, fish, shark or a mammal such as a non-human primate, mouse, rat, rabbit, gerbil, hamster, canine, feline, ovine, bovine, porcine, pachyderm, equine, or marine mammal.

The inventive dental floss compositions and related materials are of particular use on oral surfaces, such as oral tissues, as well as in oral fluids, including blood, lymph, saliva, gastric juice, and mucus, and interspaces within oral surfaces. The dental floss material may be used in the conventional manner well known in the art. Typically a section or segment of dental floss is used in an action commonly referred to as “flossing,” wherein the dental floss is passed between the contact points of two teeth and the floss is pulled or moved along the tooth surface proximate to thee gum tissue as is appropriate for the specific subject. The subject's stage of oral hygiene and/or periodontal disease will dictate the specific manner of flossing or use for the dental floss material of the present invention.

As further example, in some application such as veterinary uses or for difficult treatment in human subjects, a practitioner may prefer to wrap a segment of the dental floss material around a tooth or fix the segment temporarily in the area of the tooth, gum or other periodontal surface for some indications or situations, rather than using a typical flossing action. The inactive dental floss composition may be used with implements such as dental floss holders or applicators, as are known in the art or other aids in the use and application of the dental floss composition of the invention.

The suture compositions are useful in reducing microbial infection, adhesion, and/or contamination on biological surfaces, including, cell surfaces, membranes, mucosa, epithelia, lumenal surfaces, of a human or non-human vertebrate, including oral epithelium, or any other surface at an oral body site or site having sutured tissue. The inventive suture material may be used in the conventional manners well known in the art. A typical example includes attaching suture to a surgical needle by methods also well known in the art and passing the needle and suture through the tissue of a wound or tissue opening to create a closure or attachment or occlusion as intended by the practitioner.

The foregoing applications for the methods and compositions of the present invention are illustrative and by no means exhaustive. The invention will now be described in greater detail by reference to the following non-limiting examples. All weights are based on percent weight/volume unless otherwise clearly indicated.

### EXAMPLES

An antimicrobial assay was performed to demonstrate the inhibitory effect of LF on a variety of bacterial strains.

Preparation of LF:

A 2% (wt./vol) Im-LF/LF mixture was prepared by dissolving 2.0 g LF isolated from cow milk (DMV International Nutritionals, Veghel, The Netherlands) in a 100-ml sterile buffer solution formed of deionized water containing 1 mM EDTA (Versene NAJ from Dow Chemicals, Freeport, Tex.); 10 mM NaHCO₃ (Fisher, Fairlawn, N.J.); and 1 mM NaCl (Sigma Chemicals, St. Louis, Mo.). After adjusting the pH to 8.2 (with NaHCO₃), food-grade pectin (0.02 g; CU 201 from Herbstreith & Fox, Neurenburg, Germany) was added to the solution at room temperature with gentle stirring for ninety minutes. There resulted the partial immobilization of the dissolved LF. The formation of Im-LF was confirmed by gel filtration chromatography using Sephacryl S-200 HR column.

Preparation of LF-coated Suture Filaments

Two different suture materials, a polypropylene-type (Prolene Blue Monofilament, Ethicon Inc.) and a silk-type (Black-braided with control release, Ethicon, Inc.) were coated with the Im-LF/LF mixture as described above. Sterile suture filaments of ca.1 cm length were immersed in the Im-LF/LF mixture for 1-h, dried in sterile petri plates. The concentration of the Im-LF/LF mixture soaked into the filament material was measured using a quantitative enzyme-linked immunosorbant assay (ELISA) commercial kit for LF (Bethyl Laboratories, Montgomery, Tex.). The levels of LF in each 1-cm of soaked filament was in the range of ca. 10-200 μg.

Bacterial Test Strains:

Four different bacterial strains common to skin, skin infections, and post-operative wound infections, i.e., Staphylococcus aureus ATCC12660, Staphylococcus epidermidis ATCC12228, Pseudomonas aeruginosa ATCC27583, and Escherichia coli ATCC43895, were used for the testing. All four test strains were grown in tryptic soy broth (TSB) at 37° C.

Bacterial Growth-Inhibition Properties of LF-Treated Suture Filaments:

Bacterial growth-inhibition properties of LF coated suture filaments were measured using a microbial growth impedance detection assay (GIDA). A Bactometer® Microbial Monitoring System Model-128 (bioMerieux Vitek, Hazelwood, Mo.) was used to monitor bacterial growth by measuring impedance signals in the cultivation media.

GIDA's were performed in sixteen wells. 1-ml TSB was added to each well. Suture filaments treated with the IM-LF/LF mixture as described above were immersed in 10-mL suspensions of the bacterial test bacterial strains (containing ˜10⁴ bacteria/mL) for 10-min. Each of the filaments was gently removed from a suspension and placed into one of the wells containing TSB. Addition of untreated suture filaments, either with exposure or without exposure to one of the bacterial test strains to wells containing TSB served as controls for growth and sterility, respectively. The inoculated wells (final volume: 1-mL) were incubated at 37° C., and impedance changes in the media were monitored by the Bactometer® at 6-min intervals for 48-h. Bacterial growth curves were graphically displayed as percent changes of impedance signals versus incubation time. The amount of time required to cause a significant deviation from baseline impedance value was defined as the “detection time” (DT). The difference in DT values between growth control and test samples was considered as the “stasis” (growth-inhibition) time.

As seen in TABLE-1, all four bacterial strains demonstrated interaction (carry-through) with suture filaments. Accordingly, the suture-bound bacteria proliferated in TSB and gave an impedance signal in the GIDA. The impedance DT values ranged from 4.0 to 6.9 hours for the polypropylene sutures and LF treatment of these sutures extended the DT values in the range of 11.0 to 23.5 hours. This indicated that LF-treated sutures elicited an effective bacteriostasis ranging from +7.0 to +18.1-h. Similarly, LF treatment of silk sutures also caused bacteriostasis ranging from +7.3 to +13.9-h for the four bacterial test strains.

TABLE 1

| | Impedance detection time in hours | | | |
|---|---|---|---|---|
| | (Stasis in +hours) | | | |
| | Polypropylene suture | | Silk suture | |
| Bacterial challenge | Untreated | LF treated | Untreated | LF treated |
| S. aureus ATCC12660 | 5.6 | 11.7 (+6.1-h) | 6.4 | 13.7 (+7.3-h) |
| S. epidermidis ATCC12228 | 6.9 | 15.9 (+9.0-h) | 7.2 | 17.1 (+9.9-h) |
| P. aeruginosa ATCC27853 | 5.4 | 23.5 (+18.1-h) | 5.1 | 19.0 (+13.9-h) |
| E. coli ATCC43895 | 4.0 | 11.0 (+7.0-h) | 4.7 | 14.3 (+9.6-h) |

Each data point represents an average value for quadruplicate readings and the standard deviation for each average value is less than 0.1

Bacterial Adhesion-Inhibition Properties of LF-Coated Suture Filaments:

Bacterial adhesion/inhibition properties of LF-coated suture filaments was measured using a ³H-thymidine-labeled bacterial adhesion assay. For radiolabeling of bacteria, a 50-L inoculum of overnight culture of Staphylococcus aureus ATCC12660, Staphylococcus epidermidis ATCC12228, Pseudomonas aeruginosa ATCC27583, and Escherichia coli ATCC43895 grown in TSB was re-inoculated in a sterile 10-mL of TSB tube containing ³H-thymidine (20 μci). All the four test strains were grown at 37° C. to exponential phase (about 5-7 h) to allow optimum uptake and incorporation of ³H-thymidine into their bacterial DNA. ³H-thymidine labeled bacterial cells were harvested by centrifugation at 7,500×g, washed and resuspended in phosphate buffered saline (PBS, pH 7.2). A correlation curve was generated for each test strain between the degree of thymidine labeling (scintillation counts measured as disintegration per minute; DPM), bacterial viability (measured as total viable plate counts) and total cell counts (OD measurement at 600 nm). The density of bacterial suspension was optically adjusted to 1.0 OD at 600 nm (corresponding to ˜10⁹ cells/mL) and further diluted in PBS to a final density of 10⁶ cells/mL for adhesion experiments.

LF treated suture filaments were immersed in a 10-mL suspension of ³H-thymidine-labeled test bacterial strains (containing 10⁶ bacteria/mL) for 1-min. Each of the filaments was gently removed and placed in a scintillation vial containing 2-mL homogenizer (Scintiges™, Fisher) and incubated overnight in a 50° C. water bath. After total digestion of the suture filament, 10-mL of scintillation cocktail (ScintiSafe™ Gel, Fisher) was dispensed into the vial and thoroughly mixed. After settling and clarification of the mixture, radioactivity was measured using a liquid scintillation analyzer (Tri-Carb 2100 TR®, Packard Inc.). Addition of untreated suture filaments either exposure or without exposure to ³H-thymidine-labeled test bacteria to scintillation vials served as controls for bacterial attachment and background radioactivity, respectively.

As seen in TABLE-2, treatment of sutures with LF resulted in adhesion-inhibition of all four test strains in the range of 96.2-99.4% for the polypropylene sutures and 96.3-97.6% for the silk sutures compared to their untreated counterparts.

TABLE 2

| | Radioactivity in DPM (% adhesion-inhibition) | | | | |
|---|---|---|---|---|---|
| | Polypropylene suture | | | Silk suture | |
| Bacterial challenge | Untreated | LF treated | | Untreated | LF treated |
| S. aureus ATCC12660 | 4,387 | 167 | (96.2%) | 3,465 | 119 (96.6%) |
| S. epidermidis ATCC12228 | 3,768 | 94 | (97.5%) | 2,998 | 106 (96.5%) |
| P. aeruginosa ATCC27853 | 7,921 | 51 | (99.4%) | 8,763 | 328 (96.3%) |
| E. coli ATCC43895 | 6,543 | 72 | (98.9%) | 5,939 | 145 (97.6%) |

Each data point represents an average value for quadruplicate readings

1. A filament composition having a surface for reducing microbial contamination comprising a filament material and lactoferrin.

2. The filament composition in accordance with claim 1 wherein at least some of the lactoferrin is immobilized on a biologically active substrate via the N-terminus region of the lactoferrin.

3. The filament composition in accordance with claim 2 wherein the ratio of immobilized lactoferrin to free lactoferrin is from about 1:1 to about 1:500.

4. The filament composition in accordance with claim 2 wherein the ratio of immobilized lactoferrin to free lactoferrin is from about 1:4 to about 1:100.

5. The filament composition in accordance with claim 2 wherein the ratio of immobilized lactoferrin to free lactoferrin is about 1:20.

6. The filament composition in accordance with claim 2 wherein the biologically active substrate is a protein, a polysaccharide, a nucleic acid, a nucleotide or a lipid.

7. The filament composition in accordance with claim 2 wherein the biologically active substrate is galactose-rich polysaccharide, collagen, gelatin, fibronectin, casein, mucin, heparan-sulfate, carrageenan, pectin, deoxyribonucleic acid, adenosine triphosphate or a triglyceride.

8. The filament composition in accordance with claim 2 wherein the concentration of the lactoferrin on the surface of the filament composition for reducing microbial contamination is from about 0.0001 to about 10 mg/square inch.

9. The filament composition in accordance with claim 2 wherein the concentration of the lactoferrin on the surface of the filament composition for reducing microbial contamination is from about 0.01 to about 1 mg/sq. inch.

10. The filament composition in accordance with claim 2 wherein the filament material is a monofilament or a multifilament material.

11. The filament composition in accordance with claim 2 wherein the surface of the filament composition for reducing microbial contamination has a coating containing the lactoferrin.

12. The filament composition in accordance with claim 2 wherein the filament material is covalently bonded to the lactoferrin.

13. A dental floss composition having a surface for reducing microbial contamination comprising a dental floss material and lactoferrin.

14. The dental floss composition in accordance with claim 13 wherein at least some of the lactoferrin is immobilized on a biologically active substrate via the N-terminus region of the lactoferrin.

15. The dental floss composition in accordance with claim 14 wherein the ratio of immobilized lactoferrin to free lactoferrin is from about 1:4 to about 1:100.

16. The filament composition in accordance with claim 14 wherein the biologically active substrate is a protein, a polysaccharide, a nucleic acid, a nucleotide or a lipid.

17. The filament composition in accordance with claim 14 wherein the biologically active substrate is galactose-rich polysaccharide, collagen, gelatin, fibronectin, casein, mucin, heparan-sulfate, carrageenan, pectin, deoxyribonucleic acid, adenosine triphosphate or a triglyceride.

18. The dental floss composition in accordance with claim 14 wherein the concentration of the lactoferrin on the surface of the dental floss composition for reducing microbial contamination is from about 0.0001 to about 10 mg/square inch.

19. The dental floss composition in accordance with claim 14 wherein the concentration of the lactoferrin on the surface of the dental floss composition for reducing microbial contamination is from about 0.01 to about 1 mg/sq. inch.

20. The dental floss dental composition in accordance with claim 14 wherein the dental floss material is a monofilament material, a multifilament material or a tape.

21. The dental floss composition in accordance with claim 14 wherein the dental floss material is a multifilament material.

22. The dental floss composition in accordance with claim 14 wherein the surface of the dental floss composition for reducing microbial contamination has a coating containing the lactoferrin.

23. The dental floss composition in accordance with claim 22 wherein the coating is a pH sensitive wax or polymeric coating.

24. The dental floss composition of claim 14 wherein the dental floss material has a layer of a hydrophilic polymer treated with the lactoferrin and a permeation enhancer.

25. The dental floss composition in accordance with claim 14 wherein the dental floss material is covalently bonded to the lactoferrin.

26. A suture composition having a surface for reducing microbial contamination comprising a suture material and lactoferrin.

27. The suture composition in accordance with claim 26 wherein at least some of the lactoferrin is immobilized on a biologically active substrate via the N-terminus region of the lactoferrin.

28. The suture composition in accordance with claim 27 wherein the ratio of immobilized Lactoferrin to free Lactoferrin is from about 1:4 to about 1:100.

29. The suture composition in accordance with claim 27 wherein the biologically active substrate is a protein, a polysaccharide, a nucleic acid, a nucleotide or a lipid.

30. The suture composition in accordance with claim 27 wherein the biologically active substrate is galactose-rich polysaccharide, collagen, gelatin, fibronectin, casein, mucin, heparan-sulfate, carrageenan, pectin, deoxyribonucleic acid, adenosine triphosphate or a triglyceride.

31. The suture composition in accordance with claim 27 wherein the concentration of the Lactoferrin on the surface of the suture composition for reducing microbial contamination is from about 0.0001 to about 10 mg/square inch.

32. The suture composition in accordance with claim 27 wherein the concentration of the lactoferrin on the surface of the suture composition for reducing microbial contamination is from about 0.01 to about 1 mg/sq. inch.

33. The suture composition in accordance with claim 27 wherein the suture material is a monofilament material or a multifilament material.

34. The suture composition in accordance with claim 27 wherein the suture material is a monofilament material.

35. The suture composition in accordance with claim 27 wherein the suture material is a multifilament material.

36. The suture composition in accordance with claim 27 wherein the surface of the suture composition for reducing microbial contamination has a coating containing the lactoferrin.

37. The suture composition in accordance with claim 27 wherein the dental floss material is covalently bonded to the lactoferrin.
